(19) United States
(12) Patent Application Publication
(10) Pub. No.: US 2023/0404944 A1
(43) Pub. Date: Dec. 21, 2023

(54) CANNABINOID ENCAPSULATION TECHNOLOGY

(71) Applicant: ZELIRA THERAPEUTICS OPERATIONS PTY LTD, Perth (AU)

(72) Inventors: Ryu TAKECHI, Bentley, WA (AU); John MAMO, Bentley, WA (AU); Armin MOORANIAN, Bentley,WA (AU); Maimuna MAJIMBI, Bentley, WA (AU); Emily BROOK, Bentley, WA (AU); Hani AL-SALAMI, Bentley, WA (AU)

(21) Appl. No.: 18/251,987

(22) PCT Filed: Nov. 5, 2021
(86) PCT No.: PCT/AU2021/051313

§ 371 Date: May 5, 2023

(30) Foreign Application Priority Data
Nov. 6, 2020 (AU) 2020904057

(51) Int. Cl. A61K 31/05; A61K 31/575; A61K 47/36; A61K 47/02; A61K 9/50

(52) U.S. Cl. CPC A61K 31/05; A61K 31/575; A61K 47/36; A61K 47/02; A61K 9/5089; A61K 9/5036

(57) ABSTRACT

The invention relates to a composition comprising one or more cannabinoids, a permeation enhancer, and an alginate polymer, wherein the one or more cannabinoids and permeation enhancer are encapsulated. Such compositions may be useful in the treatment of neurodegenerative disorders, where delivery of a cannabinoid to the brain is required.

### FIELD OF THE INVENTION

The present invention relates to compositions comprising encapsulated cannabinoid(s) and a permeation enhancer, optionally with one or more terpenes. Such compositions may find use in, for example, the treatment of neurodegenerative disorders which are responsive to cannabinoids.

### BACKGROUND OF THE INVENTION

The biological activity of Cannabis has been well known for at least 2,500 years. The active components of Cannabis are cannabinoids, which are defined as any compound from a Cannabis plant that exerts its effect via cannabinoid receptors. The cannabinoids most often associated with Cannabis are (—)-trans-Δ⁹-tetrahydrocannabinol (THC) and cannabidiol (CBD), although there are at least 142 others that have been identified. There are two known types of cannabinoid receptor, CB₁ and CB₂, which are located throughout the body. CB₁ receptors are predominantly found in the brain, whilst CB₂ receptors are mainly found in the immune system.

Investigations into the use of cannabinoids for the treatment of various conditions has been growing over the last few years, in part due to relaxation of laws regulating Cannabis use in many countries. Of particular relevance for the present invention is the treatment of conditions that affect the brain.

Compositions comprising CBD and/or THC have been described for the treatment of a wide variety of neurological conditions, such as: Alzheimer's disease, anorexia, attention deficit hyperactivity disorder (ADHD), anxiety, autism, depression, epilepsy, Huntington's disease, migraines, nausea, pain, Parkinson's disease, post-traumatic stress disorder (PTSD), seizures, sleep disorders, and vomiting.

CBD is of particular interest for the present invention. CBD binds to receptors CB₁, CB₂ and 5-HT_{1A} to modulate cellular activity and inhibit excitotoxicity. Studies show that repeated administration of CBD may be neuroprotective in animal models of Alzheimer's disease via decreasing microglial activation and attenuation of memory deficits. Furthermore, activation of the endocannabinoid system has been shown to preserve cerebral capillary integrity that forms the blood-brain barrier (BBB), and exerts therapeutic effects in models of diabetes.

CBD is highly lipophilic, sensitive to light and largely broken down in the duodenum contributing to extremely low oral bioavailability in plasma and tissues (approximately 6% and 1%, respectively). Whilst chronic usage of CBD is clinically well tolerated, its high volatility and instability poses limitations for use in research and adaptation as a pharmacotherapy. The high cost of using such a drug, which only has low oral bioavailability, may also be prohibitive for many in need.

There is a need for strategies that mitigate at least one of the problems outlined above, for example, being able to deliver a cannabinoid, or a mixture of cannabinoids, to the brain to treat a neurological condition, or to prolong the residence time of the cannabinoid in an organ such as the brain, or to enhance the uptake of the cannabinoid in an organ such as the brain, or to provide a cost-effective treatment for such conditions, or to protect the cannabinoid, ora mixture of cannabinoids, from light or air, or to reduce or eliminate the amount of degradation during storage or administration.

### SUMMARY OF THE INVENTION

The present invention is predicated at least in part on the discovery that a composition comprising an encapsulated cannabinoid or mixture of cannabinoids, along with a permeation enhancer, may be suitable for delivery to the brain to treat a neurological condition, or at least provide an alternative to the solutions presently available.

In one aspect, the present invention provides a composition comprising one or more cannabinoids; a permeation enhancer; and an alginate polymer; wherein the one or more cannabinoids and permeation enhancer are encapsulated.

In another aspect, the present invention provides a process for preparing a cannabinoid composition, wherein the cannabinoid composition comprises:
one or more cannabinoids;
a permeation enhancer; and
an alginate polymer;
   the process comprising:
mixing an aqueous solution of the one or more cannabinoids and the alginate polymer to form an emulsion, then casting the emulsion into an aqueous solution of a multivalent salt to form an encapsulated cannabinoid particle.

In some embodiments, the permeation enhancer is encapsulated together with the one or more cannabinoids. In other embodiments the one or more cannabinoids and permeation enhancer are encapsulated separately, the process further comprising the steps of encapsulating the permeation enhancer by mixing an aqueous solution of the permeation enhancer and the alginate polymer to form an emulsion, then casting the emulsion into an aqueous solution of a multivalent salt to form an encapsulated permeation enhancer particle, and combining the encapsulated cannabinoid particle and the encapsulated permeation enhancer particle to form the composition of the invention.

Other aspects of the present invention will become apparent to those ordinarily skilled in the art upon review of the following description of specific embodiments of the invention.

### BRIEF DESCRIPTION OF THE FIGURE

For a further understanding of the aspects and advantages of the present invention, reference should be made to the following detailed description, taken in conjunction with the accompanying drawing, in which:

FIG. 1 shows mean concentration-time curve of a cannabinoid, cannabidiol (CBD), in plasma and brain (a, c) with corresponding cumulative concentrations shown as area under curve (AUC) graphs (b, d). CBD was administered orally to mice at a dosage of 5 mg/kg according to the following formulations: encapsulated CBD (Capsule); encapsulated CBD plus encapsulated deoxycholic acid (4 mg/kg) (Capsule+DCA) and CBD with oil (Naked). For each graph, the data represents mean±SEM bars (n=3 or 4 per treatment group for each timepoint). Analysis using one-way ANOVA revealed no statistical significance, with plasma and brain data yielding p=0.07 and p=0.67, respectively.

### DETAILED DESCRIPTION OF THE INVENTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein, the term “about” encompasses small variations in the amount of the component being referred to. For example, the term “about 98”, in the context of the term “about 98:2” means “from 97.5 to 98.4 parts of the first component compared to the second component”. As such, small variations on the above ratios are within the scope of the invention. In other contexts, it may be appropriate that the term “about” means a quantity, level, value, dimension, size, or amount that varies by as much as 15% or 10% to a reference quantity, level, value, dimension, size, or amount. The skilled artisan will be able to discern the meaning of the term “about” in keeping with the spirit of the invention.

As used herein, the term “and/or” means that the integer(s) or step(s) before the term may include the integer(s) or step(s) appearing after the term, in accordance with the usual definition of the term “and”. This term may also mean that the integer(s) or step(s) after the term may be included as an alternative to the integer(s) or step(s) appearing before the term, in accordance with the usual definition of the term “or”.

### DESCRIPTION OF EMBODIMENT(S)

In one aspect, the present invention provides a composition comprising one or more cannabinoids, a permeation enhancer, and an alginate polymer, wherein the one or more cannabinoids and permeation enhancer are encapsulated.

The advantage of compositions of the present invention are that uptake of the one or more cannabinoids by the brain is greatly enhanced compared to compositions that do not contain a permeation enhancer. Other advantages of encapsulated compositions such as the present invention are that the one or more cannabinoids and/or the permeation enhancer are greatly stabilised against degradation from light or air. Furthermore, if the one or more cannabinoids or permeation enhancer are volatile, encapsulation will reduce losses of these materials from a composition, due to the barrier nature of the encapsulated materials.

As used herein, the term “cannabinoid” means any molecule that has activity involving the endocannabinoid system. Cannabinoids may be naturally occurring, for example, found in Cannabis plants or they may be synthetically produced, for example, derivatives of naturally occurring cannabinoids. Examples of synthetic cannabinoids include nabilone and rimonabant.

To date, over 100 cannabinoids have been identified in Cannabis plants. A comprehensive list of these cannabinoids may be found in Mahmoud A. El Sohly and Waseem Gul, “Constituents of Cannabis Sativa.” (Handbook of Cannabis Roger Pertwee (Ed.) Oxford University Press, 2014; ISBN: 9780199662685).

The one or more cannabinoids of the present invention may be selected from the group consisting of: cannabigerol (E)-CBG-05, cannabigerol monomethyl ether (E)-CBGM-C5 A, cannabigerolic acid A (Z)-CBGA-C5 A, cannabigerovarin (E)-CBGV-C3, cannabigerolic acid A (E)-CBGA-C5 A, cannabigerolic acid A monomethyl ether (E)CBGAM-C5 A, cannabigerovarinic acid A (E)-CBGVAC3A, (±)-cannabichromene CBC—C5, (±)-cannabichromenic acid A CBCA-C5 A, (±)-cannabivarichromene, (±)-cannabichromevarin CBCV—C3, (±)-cannabichromevarinic acid A CBCVA-C3 A; (−)-cannabidiol CBD-05, cannabidiol momomethyl ether CBDM-05, cannabidiol-C4 CBD-C4, (−)-cannabidivarin CBDVC3, cannabidiorcol CBD-C1, cannabidiolic acid CBDA-C5, cannabidivarinic acid CBDVA-C3; cannabinodiol CBND-05, cannabinodivarin CBVD-C3; Δ⁹-tetrahydrocannabinol Δ⁹-THC—C5, Δ⁹-tetrahydrocannabinol-C4 Δ⁹-THC—C4, Δ⁹-tetrahydrocannabivarin Δ⁹-THCV—C3, Δ⁹-tetrahydrocannabiorcol Δ⁹-THCO—C1, Δ⁹-tetrahydrocannabinolic acid A Δ⁹-THCA-C5 A, Δ⁹-tetrahydrocannabinolic acid B Δ⁹-THCA-05 B, Δ⁹-tetrahydrocannabinolic acid-C4 A and/or B Δ⁹-THCA-C4 A and/or B, Δ⁹-tetrahydro-cannabivarinic acid A Δ⁹-THCVA-C3 A, Δ⁹-tetrahydrocannabiorcolic acid A and/or B Δ⁹-THCOA-C1 A and/or B, (−)-Δ⁸-trans-(6aR,10aR)-Δ⁸-tetrahydrocannabinol Δ⁸-THC—C5, (−)-Δ⁸-trans-(6aR,10aR)-tetrahydrocannabinolic acid A Δ⁸-THCA-C5 A, (−)-(6aS,10aR)-Δ⁹-tetrahydrocannabinol (−)-cis-Δ⁹-THC—C5; cannabinol CBN-05, cannabinol-C4 CBN—C4, cannabivarin CBV—C3, cannabinol C2 CBN—C2, cannabiorcol CBN—C1, cannabinolic acid A CBNA-C5 A, cannabinol methyl ether CBNM-05, (−)-(9R,10R)-trans-cannabitriol (−)-trans-CBT-05, (+)-(9S,10S)-cannabitriol (+)-trans-CBT-05, (±)-(9R,10S/9S,10R)—); cannabitriol (±)-cis-CBT-05, (−)-(9R,10R)-trans-10-O-ethyl-cannabitriol (−)-trans-CBT-OEt-05, (±)-(9R,10R/9S,10S)-cannabitriol-C3 (±)-trans-CBT-C3, 8,9-dihydroxy-Δ6a(10a)-tetrahydrocannabinol 8,9-di-OH—CBT-05, cannabidiolic acid A cannabitriol ester CBDA-C5 9-OH—CBT-05 ester, (−)-(6aR,9S,10S,10aR)-9,10-dihydroxyhexahydrocannabinol, cannabiripsol, cannabiripsol-C5, (−)-6a,7,10a-trihydroxy-Δ⁹-tetrahydrocannabinol (−)-cannabitetrol, 10-oxo-Δ^{6a}(10a)tetrahydrocannabinol OTHC); (5aS,6S,9R,9aR)-cannabielsoin CBE-05, (5aS,6S,9R,9aR)-C3-cannabielsoin CBE-C3, (5aS,6S,9R,9aR)-cannabielsoic acid A CBEA-C5 A, (5aS,6S,9R,9aR)-cannabielsoic acid B CBEA-05 B; (5aS,6S,9R,9aR)-C3-cannabielsoic acid B CBEA-C3 B, cannabiglendol-C3 OH-iso-HHCV—C3, dehydrocannabifuran DCBF-05, cannabifuran CBF-05), (−)-Δ⁷-trans-(1R,3R,6R)-isotetrahydrocannabinol, (±)-Δ⁷-1,2-cis-(1R,3R,6S/1S,3S,6R)-isotetrahydrocannabivarin, (−)-Δ⁷-trans-(1R,3R,6R)-isotetrahydrocannabivarin; (±)-(1aS,3aR,8bR,8cR)-cannabicyclol CBL-05, (±)-(1aS,3aR,8bR,8cR)-cannabicyclolic acid A CBLA-C5 A, (±)-(1aS,3aR,8bR,8cR)-cannabicyclovarin CBLV-C3; cannabicitran CBTC5; cannabichromanone CBCN—C5, cannabichromanone-C3 CBCN—C3, and cannabicoumaronone CBCON—C5, or any combination thereof.

In some embodiments, one or more cannabinoids may be encapsulated with or without one or more terpenes, together with a permeation enhancer. This ensures that the one or more cannabinoids, one or more terpenes, and permeation enhancer are delivered in the correct ratio, and may simplify the manufacturing process.

In some embodiments, the one or more cannabinoids may be encapsulated separately to the permeation enhancer. The one or more terpenes, if present, may be encapsulated with the one or more cannabinoids, or with the permeation enhancer. In some embodiments, the one or more cannabinoids, one or more terpenes, and permeation enhancer may be encapsulated separately to each other. An advantage of encapsulating components separately is that for small sized particles, each particle may be formulated with a higher amount of the active component. Providing the components in separate particles may also simplify the formulation process, to achieve the right ratio of cannabinoid to terpene to permeation enhancer.

In some embodiments, the one or more cannabinoids are selected from the group consisting of THC, THCV, CBD, CBN, CBG, CBC, and CBND, or any combination thereof. In one embodiment, the cannabinoid is CBD.

It will be appreciated by the skilled person that cannabinoids are lipophilic in nature. It is not the intention of the inventors to limit the invention to a specific cannabinoid or combination of cannabinoids, as the encapsulation technology described herein would be expected to work with any cannabinoid.

In some embodiments, the one or more cannabinoids may be present in a composition further comprising one or more terpenes, such as those that are produced naturally alongside cannabinoids in a Cannabis plant. Terpenes are derived from units of isoprene, which has the molecular formula C₅H₈. The basic molecular formula of terpenes are multiples of the isoprene unit, i.e. (C₅H₈)ₙ, where n is the number of linked isoprene units. Terpenoids are terpene compounds that have been further metabolised in the plant, typically through an oxidative process, and therefore usually contain at least one oxygen atom. The term terpene has been used in this specification to include terpenoid.

A large number of terpenes have been identified in Cannabis extracts, including monoterpenes, monoterpenoids, sesquiterpenes and sesquiterpenoids. In some embodiments, the one or more cannabinoids may be present in a composition further comprising one or more terpenes selected from the group consisting of: alloaromadendrene, allyl hexanoate, benzaldehyde, (Z)-a-cis-bergamotene, (Z)-a-trans-bergamotene, β-bisabolol, epi-a-bisabolol, β-bisabolene, borneol (camphol), cis-y-bisabolene, bomeol acetate (bomyl acetate), α-cadinene, camphene, camphor, cis-carveol, caryophyllene (β-caryophyllene), α-humulene (α-caryophyllene), γ-cadinene, Δ-3-carene, caryophyllene oxide, 1,8-cineole, citral A, citral B, cinnameldehyde, α-copaene (aglaiene), γ-curcumene, β-cymene, β-elemene, γ-elemene, ethyl decdienoate, ethyl maltol, ethyl propionate, ethylvanillin, eucalyptol, α-eudesmol, β-eudesmol, γ-eudesmol, eugenol, cis-β-famesene ((Z)-β-farnesene), trans-α-farnesene, trans-β-famesene, trans-γ-bisabolene, fenchone, fenchol (norbomanol, β-fenchol), geraniol, α-guaiene, guaiol, methyl anthranilate, methyl salicylate, 2-methyl-4-heptanone, 3-methyl-4-heptanone, hexyl acetate, ipsdienol, isoamyl acetate, lemenol, limonene, d-limonene (limonene), linolool (linalyl alcohol, β-linolool), α-longipinene, menthol, γ-muurolene, myrcene (β-myrcene), nerolidol, trans-nerolidol, nerol, β-ocimene (cis-ocimene), octyl acetate, α-phellandrene, phytol, α-pinene (2-pinene), β-pinene, pulegone, sabinene, cis-sabinene hydrate (cis-thujanol), β-selinene, α-selinene, γ-terpinene, terpinolene (isoterpine), terpineol (α-terpineol), terpineol-4-ol, α-terpinene (terpilene), α-thujene (origanene), valencene, vanillin, viridiflorene (ledene), and α-ylange, or any combination thereof.

In some embodiments, the cannabinoid is in the form of a composition comprising more than one cannabinoid. In some embodiments, the cannabinoid is in the form of a composition comprising one or more cannabinoids and one or more terpenes. In some embodiments, the one or more cannabinoids are in the form of a plant extract, such as a Cannabis extract. In other embodiments, the cannabinoid is a single cannabinoid, naturally occurring and isolated from an extract or synthetically produced. In yet other embodiments, the cannabinoid is in the form of a composition that comprises one or more cannabinoids and one or more terpenes but is not a plant extract, for example, a composition where specific cannabinoids and specific terpenes are selected and mixed in desired amounts to form the composition.

As used herein, the term “permeation enhancer” means any substance that improves or enhances penetration into an organ which has a barrier. Examples of barriers include the blood-brain barrier (BBB), the blood-cerebrospinal fluid barrier (BCSFB), retina, and small intestine. In some embodiments, the permeation enhancer is a bile acid.

Bile acids have been described as permeation enhancers for permeation into an organ or tissue. Bile acids are steroidal structures made from cholesterol in the liver (primary bile acids) or colon (secondary bile acids). There are many known bile acids, such as described in Bile acids—chemistry, biosynthesis, analysis, chemical and metabolic transformations and pharmacology, (Eds Momir Mikov and John Paul Fawcett, 2007; ISBN: O-12-045783-2).

Bile acids that may improve BBB permeability include salts, esters, and free acids of deoxycholic acid (DCA) and keto derivatives of cholic acid, such as 3α,7α-dihydroxy-12-keto-5α-cholic acid. Deoxycholic acid is a metabolite of chenodeoxycholic acid. The structures of deoxycholic acid and 3α,7α-dihydroxy-12-keto-5α-cholic acid are shown below:

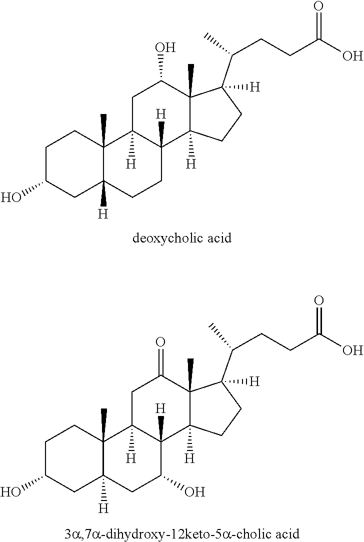

It is not the intention of the inventors to limit the invention by a specific bile acid, as any bile acid that enhances permeation of a membrane is expected to work. Bile acids have amphiphilic properties and primarily function as surfactants in the body, promoting aqueous solubility and increased fluidity of phospholipid membranes. In one embodiment, the bile acid is deoxycholic acid.

In some embodiments, the permeation enhancer provides permeation into the brain.

As used herein, the term “alginate polymer” means any alginate polymer that is in the form of a salt, for example, a sodium salt.

Alginate polymers are polysaccharide polymers typically found in brown algae or the biofilm from some bacteria such as Pseudomonas aeruginosa. The polymer is comprised of two monomers, (1-4)-linked β-D-mannuronate (M) and α-L-guluronate (G), covalently linked together in different sequences or blocks. For example, the monomers may appear in consecutive blocks of M or G, or the M and G monomers may alternate. Different algae or bacteria will produce alginate polymers of differing constitution. The M and G residues in algae or bacteria, which form the alginate polymer, may be in the form of free acids, or in the form of salts, for example, monovalent salts such as sodium salts, or potassium salts, or any combination thereof. The chemicals used in the extraction process to obtain the alginate from its biological source may determine the salt form of the alginate. For example, when using a sodium-containing chemical in the extraction process, such as sodium hydroxide, the alginate polymer produced will predominantly be in the sodium form.

Sodium alginates are commercially available in a number of forms, and their constitution will vary depending on the source of the alginate and the process used to extract and purify the alginate. For example, the sodium alginate may be low viscosity sodium alginate or medium viscosity sodium alginate. Potassium alginates are also known in the art. It is not the intention of the inventors to limit the invention to a particular type or source of alginate, as any alginate which may form a barrier layer will be suitable. In one embodiment, the alginate polymer is sodium alginate.

As used herein, the term “encapsulated” means that a substance such as a cannabinoid, with or without a terpene or permeation enhancer, is mixed with an alginate polymer, and the resulting mixture is formed into a particle and treated to stabilise the alginate polymer. Encapsulated particles may be defined as beads or capsules. The term “particle” means a bead or capsule, which may typically be spherical in shape, which has one or more substance distributed throughout the matrix. There may be no distinctive core or shell sections, or there may be a defined core comprising one or more substance, surrounded by a shell structure made of a different substance.

Without wishing to be bound by theory, stabilisation may involve an exchange reaction between sodium ions in the alginate polymer with multivalent ions such as Ca²⁺ or Ba²⁺ to form a highly cross-linked alginate barrier layer. The alginate may form cross-links upon exposure to the multivalent ion to form a stable hydrogel polymer under mild aqueous conditions. Depending upon the size of the particle, and the conditions used to exchange out, for example, sodium ions with calcium ions, a particle may be formed which has sodium alginate in the core and calcium alginate as a shell, or the calcium alginate may be present throughout the particle. Whether the sodium ions have been partially or completely replaced with the multivalent ions is of little consequence for the present invention, as both situations involve formation of an alginate which has been stabilised against degradation due to cross-linking.

As used herein, the term “tₘₐₓ” means the time required for a substance such as a cannabinoid to reach a maximum concentration in a target tissue, such as the brain.

As used herein, the term “brain penetration” means that an active compound, such as one or more cannabinoids, has been absorbed into the blood stream, has passed through the blood-brain barrier, and has been delivered to the brain.

In some embodiments, the tₘₐₓ of the one or more cannabinoids in brain tissue is less than 1 hour. The tₘₐₓ is a measure of the amount of time taken to achieve the maximum concentration of a cannabinoid in brain tissue and are typically in the order of minutes or hours, following oral consumption of the one or more cannabinoids. For example, the tₘₐₓ of the one or more cannabinoids in brain tissue is less than 1 h, or less than 0.9 h, or less than 0.8 h, or less than 0.7 h, or less than 0.6 h, or less than 0.5 h, or less than 0.4 h, or less than 0.3 h, or less than 0.2 h, or less than 0.1 h. In one embodiment, the tₘₐₓ of the one or more cannabinoids in brain tissue, when administered encapsulated with encapsulated DCA is about 0.7 h, wherein the cannabinoid is CBD.

In some embodiments, the molar ratio of the one or more cannabinoids to permeation enhancer may be from about 20:1 to about 1:20, or any rational number in between, such as for example 3.13:1. If there is more than one cannabinoid, the molar ratio of the sum of the moles of the cannabinoids, compared to the number of moles of permeation enhancer, is used. The molar ratio of the one or more cannabinoids to permeation enhancer may be in the range of from about 18:1 to about 1:1, or from about 16:1 to about 1:1, or from about 14:1 to about 1:1, or from about 12:1 to about 1:1, or from about 10:1 to about 1:1, or from about 8:1 to about 1:1, or from about 6:1 to about 1:1, or from about 5:1 to about 1:1, or from about 4:1 to about 1:1, or from about 3:1 to about 1:1, or from about 2:1 to about 1:1, or from about 1:1 to about 18:1, or from about 1:1 to about 16:1, or from about 1:1 to about 14:1, or from about 1:1 to about 12:1, or from about 1:1 to about 10:1, or from about 1:1 to about 8:1, or from about 1:1 to about 6:1, or from about 1:1 to about 5:1, or from about 1:1 to about 4:1, or from about 1:1 to about 3:1, or from about 1:1 to about 2:1. In one embodiment, the molar ratio of the one or more cannabinoids to permeation enhancer is about 3:1, wherein the cannabinoid is CBD.

In some embodiments, the encapsulated particle comprising one or more cannabinoids, with or without one or more terpenes or permeation enhancer, encapsulated cannabinoid particle (with or without one or more terpenes), or encapsulated permeation enhancer particle, has a size in the range of between about 100±50 μm and about 2000±50 μm, immediately after drying. If the one or more cannabinoids (with or without one or more terpenes) are encapsulated separately to the permeation enhancer, the particles may be the same size or they may be different. The most suitable size for particles relates to the amount of active ingredient(s) being encapsulated per particle, and the size of the subject being treated. In particular, smaller particles are more suitable for smaller subjects such as mice, as they can be easily swallowed without being chewed. Larger particles may be more suitable for humans, as they will still be able to be swallowed, but may allow a greater volume of active ingredient(s) per particle. For example the size of the particles may be between about 150±50 μm and about 2000±50 μm, between about 200±50 μm and about 2000±50 μm, between about 250±50 μm and about 2000±50 μm, between about 300±50 μm and about 2000±50 μm, between about 350±50 μm and about 2000±50 μm, between about 400±50 μm and about 2000±50 μm, between about 450±50 μm and about 2000±50 μm, between about 500±50 μm and about 2000±50 μm, between about 100±50 μm and about 1900±50 μm, between about 100±50 μm and about 1800±50 μm, between about 100±50 μm and about 1700±50 μm, between about 100±50 μm and about 1600±50 μm, between about 100±50 μm and about 1500±50 μm, between about 100±50 μm and about 1400±50 μm, between about 100±50 μm and about 1300±50 μm, between about 100±50 μm and about 1200±50 μm, between about 100±50 μm and about 1100±50 μm, between about 100±50 μm and about 1000±50 μm, between about 100±50 μm and about 900±50 μm, between about 100±50 μm and about 800±50 μm, between about 100±50 μm and about 700±50 μm, between about 100±50 μm and about 600±50 μm, between about 100±50 μm and about 500±50 μm, between about 100±50 μm and about 400±50 μm, between about 100±50 μm and about 300±50 μm, and between about 100±50 μm and about 200±50 μm. In one embodiment, the encapsulated cannabinoid particle or encapsulated permeation enhancer particle has a size of about 400±50 μm, wherein the cannabinoid is CBD and/or the permeation enhancer is DCA.

In another aspect, the present invention provides a process for preparing a cannabinoid composition, wherein the cannabinoid composition comprises:
one or more cannabinoids;
a permeation enhancer; and
an alginate polymer;
   the process comprising:
   mixing an aqueous solution of the one or more cannabinoids and the alginate polymer to form an emulsion, then casting the emulsion into an aqueous solution of a multivalent salt to form an encapsulated cannabinoid particle.

In one embodiment, the process for preparing a cannabinoid composition requires the one or more cannabinoids and the permeation enhancer to be mixed, together with the alginate polymer, to form an emulsion, and then the emulsion is cast so that the one or more cannabinoids and permeation enhancer are encapsulated together following exposure to a multivalent salt.

In another embodiment, the process for preparing a cannabinoid composition requires the one or more cannabinoids are mixed together with the alginate polymer to form an emulsion, and the permeation enhancer is mixed together with the alginate polymer to form another emulsion, and then the emulsions are cast separately so that the one or more cannabinoids and permeation enhancer are encapsulated, separately, following exposure to a multivalent salt.

In some embodiments, if a terpene is present it may be encapsulated with the one or more cannabinoids and permeation enhancer when they are encapsulated together. In other embodiments, if a terpene is present, it may be encapsulated with the one or more cannabinoids or encapsulated with the permeation enhancer, if they are encapsulated separately.

The multivalent salt used to form encapsulated particles may be any salt that is able to exchange a monovalent cation such as sodium or potassium, in the alginate polymer with a multivalent cation. Typical cations used may be calcium or barium. The counter-anion may be any anion that is suitable to be used in an aqueous solution. Typical counter-anions are halides or nitrates. Halides may be fluorides, chlorides, bromides, or iodides. The suitability of any such multivalent salt will depend on a number of factors such as aqueous solubility of the salt, availability, and price. In one embodiment, the multivalent salt is calcium chloride.

In some embodiments, the one or more cannabinoids may be provided as a solution dissolved in an organic soluble oil. The one or more cannabinoids may be dissolved with or without one or more terpenes. For example, it may be convenient to provide the one or more cannabinoids dissolved in an oil which has been used to extract the one or more cannabinoids, such as from a Cannabis plant. Extracting oils may be any edible oil, and may be selected from the group consisting of olive oil, hemp oil, sesame oil, coconut oil, vegetable oil, canola oil, grape seed oil, almond oil, and medium chain triglyceride (MCT) oil, or a combination thereof. In some embodiments, the one or more cannabinoids may be provided without an oil, as a solid powder. In one embodiment, the one or more cannabinoids are provided as a solution dissolved in medium chain triglyceride.

In another aspect, the present invention provides a process for preparing a cannabinoid composition, wherein the cannabinoid composition comprises:
one or more cannabinoids;
a permeation enhancer; and
an alginate polymer;
   the process comprising:
mixing an aqueous solution of the one or more cannabinoids and the alginate polymer to form a first emulsion, then casting the first emulsion into an aqueous solution of a multivalent salt to form an encapsulated cannabinoid particle;
mixing an aqueous solution of the permeation enhancer and the alginate polymer to form a second emulsion, then casting the second emulsion into an aqueous solution of a multivalent salt to form an encapsulated permeation enhancer particle; and
mixing the encapsulated cannabinoid particle and the encapsulated permeation enhancer particle to form the encapsulated cannabinoid composition.

In another aspect, the present invention provides a method of enhancing brain permeation of one or more cannabinoids, comprising administering to a subject an effective amount of a composition as described above.

In some embodiments, the half-life (t_{1/2}) of the one or more cannabinoids in brain tissue is more than 1 hour. The half-life is a measure of the amount of time taken for the amount of the one or more cannabinoids to be reduced to half of its initial value. For example, the half-life of the one or more cannabinoids in brain tissue is greater than 1 h, or greater than 1.5 h, or greater than 2 h, or greater than 2.5 h, or greater than 3 h, or greater than 3.5 h, or greater than 4 h, or greater than 4.5 h, or greater than 5 h, or greater than 5.5 h. In one embodiment, the half-life of the one or more cannabinoids in brain tissue, when administered encapsulated with encapsulated DCA is about 4.5 h, especially wherein the cannabinoid is CBD.

In some embodiments, the brain permeation of the cannabinoid is enhanced due to the composition comprising one or more cannabinoids which may be encapsulated together with or separately to a permeation enhancer. The one or more cannabinoids may be encapsulated with or without one or more terpenes. The amount of enhancement may be quantified by comparing the total amount of the one or more cannabinoids accumulated in the brain when delivered via encapsulated cannabinoid and permeation enhancer (encapsulated either together or separately, and with or without one or more terpenes), versus the total amount of one or more cannabinoids accumulated in the brain when delivered via encapsulated cannabinoid with no permeation enhancer, or via non-encapsulated cannabinoid with no permeation enhancer. The amount of cannabinoid may be quantified, for example, by measuring the Cₘₐₓ, AUC₀₋ₜ, or AUC_{0-inf_obs}. Cₘₐₓ is a measure of the maximum concentration of a cannabinoid attained in brain tissue after consumption of the cannabinoid, regardless of the amount of time taken to reach this concentration. AUC_{0_t} and AUC_{0-inf_obs} are a measure of the area under the curve from zero to a certain time point (t) or from zero to observed infinity (inf_obs), when the amount of cannabinoid present in the brain is plotted against time, following consumption of the cannabinoid.

In some embodiments, the amount of enhancement of the one or more cannabinoids in the brain, based on Cₘₐₓ, may be within a range of between about 200% and about 300%, such as between about 210% and about 300%, or between about 220% and about 300%, or between about 230% and about 300%, or between about 240% and about 300%, or between about 250% and about 300%, or between about 260% and about 300%, or between about 270% and about 300%, or between about 200% and about 290%, or between about 200% and about 280%, or between about 200% and about 270%, or between about 200% and about 260%, when the one or more cannabinoids are administered encapsulated with a permeation enhancer versus encapsulated and without permeation enhancer. In one embodiment, when the cannabinoid is encapsulated CBD, the amount of enhancement of CBD in the brain, based on Cₘₐₓ, is about 273% when administered in encapsulated form separately to encapsulated DCA, versus encapsulated and without DCA.

In some embodiments, the amount of enhancement of the one or more cannabinoids in the brain, based on Cₘₐₓ, may be within a range of between about 100% and about 200%, such as between about 110% and about 200%, or between about 120% and about 200%, or between about 130% and about 200%, or between about 140% and about 200%, or between about 150% and about 200%, or between about 160% and about 200%, or between about 100% and about 190%, or between about 100% and about 180%, or between about 100% and about 170%, or between about 100% and about 160%, or between about 100% and about 150%, or between about 100% and about 140%, when the one or more cannabinoids are administered encapsulated with a permeation enhancer versus non-encapsulated and without permeation enhancer. In one embodiment, when the cannabinoid is encapsulated CBD, the amount of enhancement of CBD in the brain, based on Cₘₐₓ, is about 149% when administered in encapsulated form separately to encapsulated DCA, versus non-encapsulated and without DCA.

In some embodiments, the amount of enhancement of the one or more cannabinoids in the brain, based on AUC_{0_t}, may be within a range of between about 200% and about 300%, such as between about 210% and about 300%, or between about 220% and about 300%, or between about 230% and about 300%, or between about 240% and about 300%, or between about 200% and about 290%, or between about 200% and about 280%, or between about 200% and about 270%, or between about 200% and about 260%, or between about 200% and about 250%, or between about 200% and about 240%, or between about 200% and about 230%, when the one or more cannabinoids are administered encapsulated with a permeation enhancer versus encapsulated and without permeation enhancer. In one embodiment, when the cannabinoid is encapsulated CBD, the amount of enhancement of CBD in the brain, based on AUC_{0_t}, is about 237% when administered in encapsulated form separately to encapsulated DCA, versus encapsulated and without DCA.

In some embodiments, the amount of enhancement of the one or more cannabinoids in the brain, based on AUC_{0_t}, may be within a range of between about 100% and about 200%, such as between about 110% and about 200%, or between about 120% and about 200%, or between about 130% and about 200%, or between about 140% and about 200%, or between about 150% and about 200%, or between about 160% and about 200%, or between about 170% and about 200%, or between about 180% and about 200%, or between about 100% and about 190%, or between about 100% and about 180%, or between about 100% and about 170%, or between about 100% and about 160%, when the one or more cannabinoids are administered encapsulated with a permeation enhancer versus non-encapsulated and without permeation enhancer. In one embodiment, when the cannabinoid is encapsulated CBD, the amount of enhancement of CBD in the brain, based on AUC_{0_t}, is about 178% when administered in encapsulated form separately to encapsulated DCA, versus non-encapsulated and without DCA.

The present invention will now be more fully described by reference to the following non-limiting Examples.

Starting Materials

CBD (14.5% w/w solubilised extract in Miglyol 812N), as a representative cannabinoid, and Miglyol 812N were provided by Zelira Therapeutics (Perth, Western Australia). Medium viscosity sodium alginate (MVSA), deoxycholic acid (DCA) and calcium chloride (anhydrous, 98%) were purchased from Sigma-Aldrich (St Louis, MO, USA). Formulations were made up in HPLC grade deionised water.

### Example 1— Preparation of Encapsulated Cannabinoid Formulation

A solution containing 1.5% w/v MVSA in 80 mL of HPLC-grade deionised water was mixed overnight. CBD (109.24 mg in 800 μL of Miglyol 812N) was added to the MVSA solution while protected from air and light, and then mixed for 3 days to produce a cannabinoid-alginate formulation. Separately, DCA (10 mg) was added to a solution of MVSA (1.5% w/v in 80 mL of HPLC-grade deionised water) and mixed overnight. All mixtures were stirred at the same speed at room temperature. Complete emulsification was noted by either clouding of the mixture or absence of powder suspensions.

The CBD and DCA formulations were encapsulated separately, immediately after emulsification, using a vibrating Encapsulator B-390 (BUCHI Labortechnik, Switzerland), under the following conditions: frequency range 2000 Hz; air pressure 950 mbar; 200 μm nozzle; and flow regulating valve set at 2 rotations from the tightest starting point.

Prepared formulations were cast into a 100 mM CaCl₂ bath, which was stirred with a mild vortex, at a flow rate of 5 mL/minute, to form spherical particles. After 10 minutes in the calcium chloride bath, particles were sieved, rinsed with deionised water and dried with a paper towel patted under the strainer. The particles were placed on a petri dish, covered and dried completely at 37° C. for 2.5 days, to produce CBD or DCA in a matrix of sodium alginate, and encapsulated with a coating of calcium alginate. The encapsulated particles were analysed and used for experimentation within 48 hours of drying.

Characterisation of Encapsulated CBD

The CBD loading and particle size were determined by using HPLC (Prominence, Shimadzu LC-20AT liquid chromatographer, SIL-20A autosampler and SPD-20A-UV/Vis detector, Japan) and a particle analyser (Zetasizer 3000 HS and Mastersizer 2000, Malvern Instruments, Malvern, UK), respectively. Briefly, 5 mg of encapsulated particles were agitated and suspended in PBS (pH 8.5) for 20 h and centrifuged for 15 min at 13200 rpm at 10° C. The supernatant was collected and diluted with a mobile phase mixture (acetonitrile:water=75:25). The liberated CBD was determined according to a previously described HPLC protocol (Mooranian et al., Bile acid bio-nanoencapsulation improved drug targeted-delivery and pharmacological effects via cellular flux: 6-months diabetes preclinical study. Sci Rep. 2020, 10, 106). Encapsulation efficiency was determined with published formulae as the relationship between theoretical and confirmed CBD loading within the particles (\Nagle et al., Micro-nano formulation of bile-gut delivery: rheological, stability and cell survival, basal and maximum respiration studies. Sci. Rep. 2020, 10, 7715), in accordance with the following formulae:

$$Drug loading ( \% ) = \frac{drug in particle ( mg )}{weight of particle ( mg )} \times 100 \\ Encapsulation efficiency ( \% ) = \frac{drug content}{theoretical content} \times 100$$

Formation of DCA particles in 1.5% MVSA was consistent across all attempts. The particles were spherical and their size was 400±50 μm immediately after drying, which was considered suitable for oral consumption in mice. The CBD particles were milky white and their appearance remained consistent for 30 days at room temperature; followed by gradual discoloration over longer time periods with the particles becoming brown, rough and shrivelled. Following the desiccation process, over 98% of encapsulated CBD remained intact after exposure to ambient air and light for 72 hours, indicating excellent protection of particles by encapsulation. This data demonstrates that the encapsulation protocol provides good protection of cannabinoids and permeation enhancer from exposure to the environment, which enables its prolonged use in a research setting, as well as over prolonged periods during transport and storage, in a commercial setting.

The encapsulation efficiency of CBD was calculated to be 23±1.2%, and CBD loading was estimated as 2.05±0.10%. Encapsulation efficiency is a measure of how much of a substance, such as CBD, was actually encapsulated into the particles, compared with the theoretical amount that should be encapsulated. The skilled person would be easily able to adjust the encapsulation efficiency, if desired, for example by using CBD in powder form rather than as a solution dissolved in medium-chain triglycerides.

### Example 2— In Vivo Animal Experiments

Healthy C57BL/6J wild type mice obtained from Animal Resource Centre, WA were group-housed in a temperature-controlled laboratory on a 12-hr light/dark cycle with standard chow and water provided ad libitum (14 months, female, average weight 36±3 g, n=45). The mice were fasted overnight prior to CBD administration. Experiments were conducted according to approved animal ethics protocol.

Mice were restrained and orally administered 5 mg/kg weight CBD in oil by itself (Naked group), in encapsulated particle form (Capsule group), or in encapsulated particle form with additional 4 mg/kg weight DCA particles (“Capsule+DCA” group). The cannabinoid-containing compositions were mixed in raspberry jam, which improved palatability. The same amount of raspberry jam was given to all mice to limit treatment variability. Prior to sacrifice, mice were anesthetised with isoflurane gas and blood was obtained via cardiac puncture into EDTA-coated tubes. Mice were euthanised by cervical dislocation and the brain was collected and snap frozen immediately at 0.3 h, 1 h, and 3 h post-administration. Four mice were used per time point per group, except for the Capsule and Naked groups at 1 h, where three mice were used. Plasma was collected by centrifuge (2500 g, 10 minutes, 4° C.). Samples were stored at −80° C. until analysis.

The concentration of CBD in plasma and brain tissue was determined using a previously described protocol (Galettis, Development of a simple LCMSMS method for THC and metabolites in plasma, Asia-Pac. J. Clin. Oncol. 2016, 12, 13-34). Briefly, plasma samples were thawed and 50 μL were added to 100 μL of acetonitrile containing deuterated internal standards. Brain samples (500 mg) were homogenised in methanol (500 μL) using a Tissue-Lyser with a 3 mm steel ball bearing, the samples were then centrifuged and 50 μL of homogenate was added to 100 μL of acetonitrile containing deuterated internal standards. Plasma and brain samples were vortexed then centrifuged and the supernatant was transferred to a vial and injected onto a LCMS system (Shimadzu 8060, Shimadzu, Australia). A Kinetex Biphenyl column (50×3 mm, 2.6 μm) was used with a solvent gradient, with solvents containing 0.1% formic acid, acetonitrile and water for the analysis. The calibration curve ranged from 0.5-500 ng/mL with a limit of quantitation of 0.5 ng/mL.

The results are expressed as mean and where applicable, the standard error of mean (SEM) is provided. Graphpad Prism v7 (Graphpad, Inc., USA) was used to generate concentration-time graphs on linearized log₁₀, scale and to calculate area under the concentration curve (AUC). Statistical significance between treatment groups was determined using one-way ANOVA with significance at p<0.05.

Pharmacokinetic parameters were approximated using established models in Microsoft Excel using the add-in program PKSolver (Microsoft Excel 2010) and are expressed below as median.

These included terminal half-life (t_{1/2}), maximum concentration (Cₘₐₓ), time to reach Cₘₐₓ (tₘₐₓ), area under the concentration curve from zero to a certain time (AUC₀₋ₜ) and area under the concentration curve from zero to observed infinity (AUC_{0-inf_obs}).

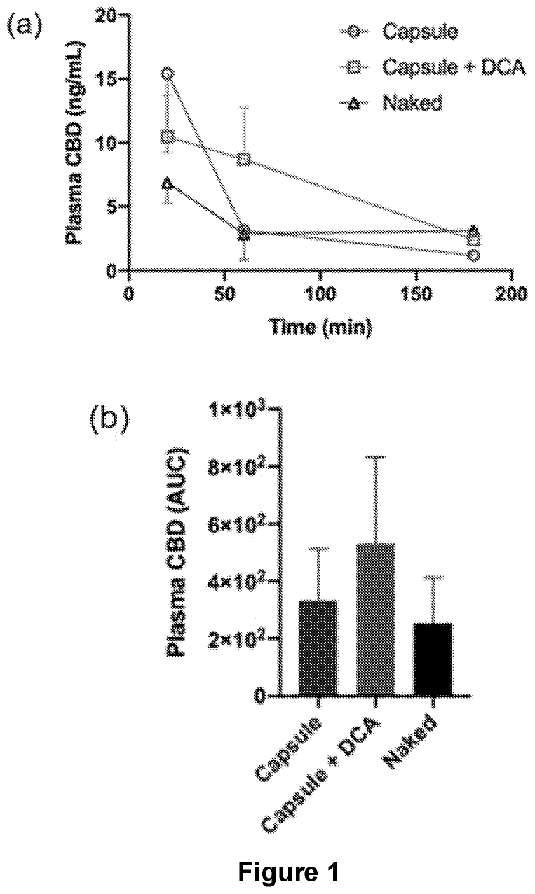

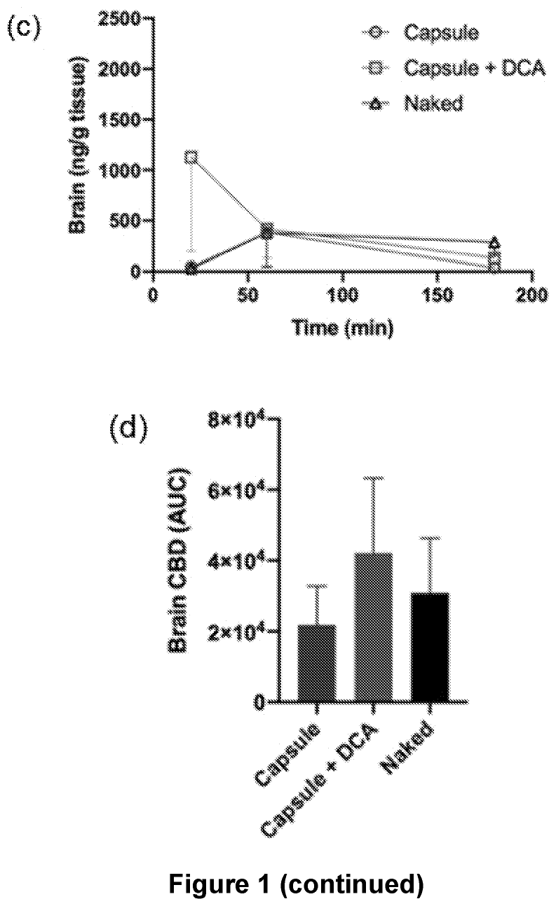

FIG. 1 illustrates the mean plasma and brain concentrations of CBD in mice administered 5 mg/kg CBD, encapsulated with or without DCA (Capsule+DCA or Capsule), compared to CBD and oil with no DCA (Naked), along with the corresponding area under concentration (AUC) graphs. Data was accrued at three time points over 3 hours to focus on the immediate effects of CBD encapsulation.

At 0.3 hours post oral administration, the plasma levels of CBD in the Capsule group was more than 2-fold higher than the Naked group (FIG. 1a), whilst that for the Capsule+DCA mice was between these two groups, likely due to higher tissue uptake. After 1 hour, the Capsule group mice showed a significant reduction of CBD levels, reaching similar plasma CBD concentrations as the Naked group. In contrast, the plasma CBD concentration of mice given Capsule+DCA surprisingly showed only a slight decrease in CBD levels, yet were substantially higher than the Capsule or Naked group mice. After 3 hours, CBD plasma levels were similar across all three groups.

Remarkably, the brain samples showed very low CBD concentrations of Capsule and Naked groups, compared to Capsule+DCA (FIG. 1c) after 0.3 hours. When CBD was given in combination with the permeation enhancer, DCA (Capsule+DCA group), the mean brain CBD concentration was 1048 ng/mg tissue, a remarkable 40- and 30-fold increase compared to the Naked and Capsule groups, respectively.

After 1 hour, and at 3 hours, brain CBD concentrations were comparable across all three groups (FIG. 1c).

The AUC graphs (FIGS. 1b and 1d) show, over 3 hour post-oral administration, the cumulative plasma concentrations of CBD was comparable between the Capsule group and the Naked group. Surprisingly, the concentration for the Capsule+DCA group mice was more than double the concentration of the other two groups (FIG. 1b). Similar results were obtained for brain concentrations of CBD after 3 hour (FIG. 1d). The trends described did not reach statistical significance using Anova (plasma: p=0.07 and brain: p=0.67), however, this may be due to the small sample sizes.

The pharmacokinetic parameters generated using MATLAB R2017a are summarised in Table 1. Plasma terminal half-life (t_{1/2}) of CBD in mice that were given CBD in oil (Naked group) was 2.2 h. The plasma CBD concentration in the Naked group mice reached its peak at 0.3 h post-oral administration, which was 7.7 ng/mL. The plasma absorption profile of CBD oil was comparable with published findings (Xu et al., Pharmacokinetics of oral and intravenous cannabidiol and its antidepressant-like effects in chronic mild stress mouse model, Environ. Toxicol. Pharmacol. 2019, 70, 103202), albeit with minimal variation in the pharmacokinetic parameters that is likely to be dose dependent. As can be seen from Table 1, plasma bioavailability as indicated by Cₘₐₓ and AUC₀₋ₜ was mildly increased for when CBD was encapsulated in alginate, compared to CBD in oil. The amount of CBD in plasma, when consumed in encapsulated form, based on Cₘₐₓ, increased by 42% (Capsule versus Naked) and 43% (Capsule+DCA versus Naked). The amount of CBD in plasma, when consumed in encapsulated form, based on AUC_{0_t}, increased by 18% (Capsule versus Naked) and 29% (Capsule+DCA versus Naked). With the same tₘₐₓ, it may be concluded that encapsulation may have a similar rate of intestinal absorption as naked oil, however it may protect against extensive first-pass metabolism to promote CBD into the surrounding capillaries.

TABLE 1
Pharmacokinetic parameters of cannabidiol (CBD) administered orally to mice
at a dosage of 5 mg/kg in the following formulations:
1) Capsule, 2) Capsule + deoxycholic acid (DCA) (4 mg/kg), and
3) Naked CBD oil formulation, in plasma and brain.

| | | Plasma | | | Brain | | |
|---|---|---|---|---|---|---|---|
| Parameter | Unit | Capsule | Capsule + DCA | Naked | Capsule | Capsule + DCA | Naked |
| t_{1/2} | h | 1.1 | 0.4 | 2.2 | 0.9 | 4.5 | ND |
| tₘₐₓ | h | 0.3 | 1.0 | 0.3 | 1.0 | 0.7 | 2.0 |
| Cₘₐₓ | ng/ml or | 10.9 | 11.0 | 7.7 | 280.9 | 1048.2 | 421.5 |
| | ng/g | | | | | | |
| AUC₀₋ₜ | ng/ml * h | 15.2 | 16.7 | 12.9 | 368.1 | 1242.1 | 447.4 |
| AUC_{0-inf}_obs | ng/ml * h | 16.3 | 6.5 | 14.9 | 75.0 | 4378.4 | ND |

Parameters were generated using standard non-compartmental analysis in PKSolver with plasma and brain concentration data. Each value is expressed as Median.
t_{1/2} = terminal half-life;
Cₘₐₓ = maximum concentration;
tₘₐₓ = time to reach Cₘₐₓ;
AUC₀₋ₜ = area under the concentration curve from zero to 3 hours post oral administration;
AUC_{0-inf}_obs = AUC from zero to observed infinity;
ND = not determined.

Interestingly, the plasma t_{1/2} of CBD was halved for the Capsule group mice compared to Naked group mice, which suggests increased clearance via excretion and/or tissue/cell uptake. The plasma t_{1/2} was even shorter in Capsule+DCA mice, indicating significantly increased CBD clearance from plasma when encapsulated DCA was present.

Consistently with the plasma results, the brain tissue analyses of CBD revealed in mice that were given Capsule CBD, the tₘₐₓ was 1 h, half of the Naked group mice, indicating significantly faster brain tissue uptake of CBD when encapsulated with alginate. Furthermore, when CBD wad administered with DCA (Capsule+DCA group) the tₘₐₓ was one third of the Naked group mice, which also resulted in significantly higher Cₘₐₓ, AUC₀₋ₜ and AUC_{0-inf_obs}. The amount of CBD in brain, based on Cₘₐₓ, increased by 273% when CBD was administered with DCA versus without (Capsule+DCA versus Capsule), and increased by 149% when administered encapsulated with DCA versus non-encapsulated and without DCA (Capsule+DCA versus Naked). Similarly, the amount of CBD in brain, based on AUC_{0_t}, increased by 237% when CBD was administered with DCA versus without (Capsule+DCA versus Capsule), and increased by 178% when administered encapsulated with DCA versus non-encapsulated and without DCA (Capsule+DCA versus Naked).

The above data reveal a remarkable increase in brain tissue uptake of cannabinoid by alginate encapsulation, when in combination with a permeation enhancer. Moreover, the mice that were administered with CBD Capsule+DCA showed substantially longer tv 2, indicating prolonged retention of CBD within the brain.

Taken together, this study indicates that encapsulation with an alginate effectively protects cannabinoids such as CBD from oxidation, degradation by light, and acidic digestion within the stomach, and enhances absorption through the GI tract and cumulative plasma bioavailability. Furthermore, it has been shown for the first time that a bile acid, such as DCA, increases uptake and retention of cannabinoids within the brain. Bile acids such as DCA may therefore significantly promote the neuroprotective efficacy of orally administered cannabinoids, particularly for the treatment of neurodegenerative disorders.

It is to be understood that, if any prior art publication is referred to herein, such reference does not constitute an admission that the publication forms a part of the common general knowledge in the art, in Australia or any other country.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A composition comprising:
one or more cannabinoids;
a permeation enhancer which is a bile acid; and
an alginate polymer;
wherein the one or more cannabinoids and permeation enhancer are encapsulated.

2. The composition according to claim 1, wherein the one or more cannabinoids and permeation enhancer are encapsulated separately.

3. The composition according to claim 1, wherein the composition has a tₘₐₓ for brain penetration of the one or more cannabinoids of less than 1 hour.

4. The composition according to claim 1, wherein the one or more cannabinoids are selected from the group consisting of THC, THCV, CBD, CBN, CBG, CBC, and CBND, or any combination thereof.

5. The composition according to claim 1, wherein bile acid is deoxycholic acid.

6. The composition according to claim 1, wherein the alginate polymer is sodium alginate.

7. The composition according claim 1, wherein the molar ratio of the one or more cannabinoids to permeation enhancer is from about 20:1 to about 1:20.

8. A process for preparing a cannabinoid composition, wherein the cannabinoid composition comprises:
one or more cannabinoids;
a permeation enhancer which is a bile acid; and
an alginate polymer;
the process comprising:
mixing an aqueous solution of the one or more cannabinoids and the alginate polymer to form an emulsion, then casting the emulsion into an aqueous solution of a multivalent salt to form an encapsulated cannabinoid particle.

9. The process according to claim 8, wherein the one or more cannabinoids and bile acid are encapsulated together.

10. The process according to claim 8, wherein the one or more cannabinoids and bile acid are encapsulated separately.

11. The process according to claim 10, further comprising the steps of:
i) mixing an aqueous solution of the bile acid and the alginate polymer to form an emulsion, then casting the emulsion into an aqueous solution of a multivalent salt to form an encapsulated bile acid particle; and
ii) mixing the encapsulated bile acid particle with the encapsulated cannabinoid particle.

12. The process according to claim 8, wherein the one or more cannabinoids are provided as a solution dissolved in medium chain triglyceride.

13. The process according to claim 8, wherein the one or more cannabinoids are selected from the group consisting of THC, THCV, CBD, CBN, CBG, CBC, and CBND, or any combination thereof.

14. The process according to claim 8, wherein the bile acid is deoxycholic acid.

15. The process according to claim 8, wherein the alginate polymer is medium viscosity sodium alginate.

16. The process according to claim 8, wherein the multivalent salt is calcium chloride.

17. The process according to claim 8, wherein the molar ratio of the one or more cannabinoids to permeation enhancer is from about 20:1 to about 1:20.

18. A method of enhancing brain permeation of one or more cannabinoids, comprising administering to a subject an effective amount of a composition according to claim 1.
